(19) United States
(12) Patent Application Publication
(10) Pub. No.: US 2007/0190110 A1
(43) Pub. Date: Aug. 16, 2007

(54) Agents and devices for providing blood clotting functions to wounds

(76) Inventors: Cornelis H. Pameijer, Simsbury, CT (US); Steven Jensen, South Jordan, UT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 11/703,491

(22) Filed: Feb. 6, 2007

Related U.S. Application Data
(60) Provisional application No. 60/772,043, filed on Feb. 10, 2006.

(51) Int. Cl. A61F 2/00

(52) U.S. Cl. 424/423

(57) ABSTRACT

Hemostatic agents and devices are made from oxidized cellulose fiber, the oxidized cellulose having a carboxylation content increased by the action of nitrogen dioxide on virgin cellulose fiber. A composition may be incorporated into the oxidized cellulose fiber to cause a pharmacological effect on a wound to which the hemostatic agents and devices are applied. When applied, the oxidized cellulose fiber causes blood emanating from the wound to clot. The oxidized cellulose fiber can either be resorbed into the wound or removed from the wound after healing. A hemostatic bandage includes a pad of unwoven oxidized cellulose fibers mounted on a substrate. Methods of arresting a flow of blood emanating from a wound using such devices are also disclosed. Methods of fabricating oxidized cellulose are also disclosed.

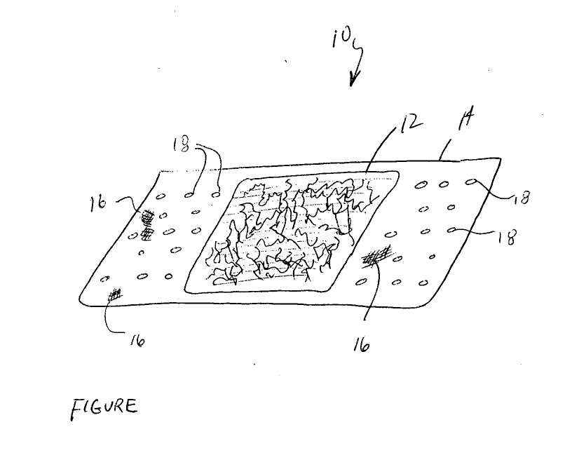

### CROSS REFERENCE TO RELATED APPLICATIONS

This application claims priority to U.S. Provisional Patent Application Ser. No. 60/772,043 for “A Device for Delivering Drugs Increasing Healing Potential,” filed Feb. 10, 2006, the contents of which are herein incorporated by reference in their entirety.

### TECHNICAL FIELD

This invention relates generally to wound healing devices and, more particularly, to devices capable of causing hemostasis at the bleed site of a wound.

### BACKGROUND OF THE INVENTION

Medical, dental, and veterinary practitioners often encounter patients with open wounds that are caused by accidents or other injuries or that are the result of surgical procedures. In the case of trauma or surgery, the presence of an open wound presents not only a risk for infection, but loss of blood can cause serious complications and in some instances death. Furthermore, uncontrolled bleeding complicates the quality and outcome of surgical procedures. After stopping the flow of blood, the principal method of treating these open wounds is to suture the adjacent defining tissue together. However, some wounds result in a gap or void in soft tissues, and in these cases suturing is not always feasible or practical.

The natural method of a body to repair an open wound in the tissue is to allow blood to fill the void that results from the wound. The blood filling such a void subsequently coagulates to form a blood clot or a soft plug, which when left undisturbed will then heal through natural organization. This blood clot or soft plug forms a barrier that inhibits the ingress of bacteria, thus preventing infection. This soft plug also contributes to the process of cell replacement during the formation of new soft and hard tissues.

These same problems are found in wounds in almost all large mammals. It is a common practice, whether treating a man or an animal, to first stop the flow of blood from a wound by applying pressure. The application of pressure will facilitate the more efficient forming of a clot. This is usually followed by protecting the clot from being prematurely dislodged and preventing the ingress of foreign bodies that would cause disease. This is usually done with the aid of surface bandages or dressings. These wounds can also be treated on the surface thereof with medications to aid in healing and reducing disease.

Open wounds, especially those in the oral cavity, create a variety of problems. For instance, during a tooth extraction a large bleeding gap or socket is created. The distance that separates the two soft tissue surfaces across the gap is typically too great to enable the two surfaces to be united as one. Thus, the sockets characteristic of tooth extractions are generally not amenable to being sutured. In the case of a tooth extraction, bacteria fill the resulting socket, which may in turn cause the tissue surrounding the socket to become breeding grounds for infections. If normal blood clotting functions occur, a soft plug coagulates in the socket and initiates the healing process. Treatment for these gap or socket-type wounds often involves counseling patients to keep this area clean without disturbing this newly formed coagulated soft plug. To encourage proper cleaning procedures, dental practitioners often provide squirt bottles as a practical means of removing debris by irrigating the wound areas. The degree of success is entirely dependent on patient compliance, and patients must execute constant vigilance in order to avoid dislodging the newly formed soft plug for several days post-extraction.

The soft plug can easily be dislodged by ordinary events that occur in the mouth every day. Events as minor as eating or sucking on a straw may dislodge this soft plug. If the soft plug were to be dislodged before healing can occur, or if there is a lack of bleeding resulting in the absence of a blood clot, a problem known as “dry socket” can occur. A dry socket can rapidly develop into an infection of the adjacent bone since the protective action of a blood clot is absent. Dry sockets are excruciatingly painful and subsequent treatment is time consuming and needs to be addressed by a dentist or other competent caregiver.

The company Upjohn markets a “sterile absorbable gelatin sponge” called GelFoam®, which is made from gelatin, a digestible food stuff. This product comes in flat sheets. When placed onto bleeding tissues (e.g., a socket-type wound), GelFoam absorbs blood like a sponge and forms a coagulum. This product is also physiologically absorbable by the body in the event it becomes trapped inside healing tissues. A disadvantage of GelFoam is that it does not withstand the oral environment. Once placed into the oral environment saliva is absorbed by the foam, thereby causing the foam to prematurely break down and become less effective.

Another disadvantage of GelFoam is the lack of physical cohesion within the material itself. Once the material contacts blood from a wound it converts to a slimy gel. This slimy gel acts like a lubricant with regard to the bleeding tissues since it does not incorporate the blood cells themselves. The resulting GelFoam plug is often delicate and easily displaced by physical means. In particular, the plug is easily removed by common events in the mouth such as eating or oral hygiene activities such as brushing teeth. In any wound gap, a GelFoam coagulated plug is not an ideal improvement over the body's own healing process.

The company Johnson & Johnson markets a knitted fabric absorbable hemostat known as Surgicel®. Surgicel is manufactured from wood pulp that contains about fifty percent cellulose by mass. The cellulose is purified via a decomposition process followed by a recomposition process. When recomposed, the cellulose is hydrolyzed and “regenerated” into what is commonly known as rayon (e.g., by treatment of the cellulose with carbon disulfide in an alkaline environment). Rayon is cellulose that is fragmented or broken at particular molecular linkages. This hydrolyzed rayon is oxidized under controlled conditions with nitrogen tetroxide to form oxidized regenerated cellulose (ORC). As the major reaction product, this ORC also includes carboxylic acid functions substituted for the functional groups on the base glucose molecules that make up the cellulose. Additionally, the reaction of the cellulose with nitrogen tetroxide at the fragmented molecular linkages also causes a number of additional reaction products to form, namely ORC products having two and three ketone groups substituted for the functional groups on the glucose molecule. The ORC products having substituted ketone groups have been found to be controlling with respect to degradation of the ORC in the body such that the biological absorption of the body is related to the ketones.

A disadvantage of Surgicel, however, is that the ketone-substituted ORC molecules are needed to facilitate the absorption of the carboxylic acid-substituted ORC molecules. Cellulose itself cannot be absorbed into the body and broken down because of the biological nature of the tissue of the body. Accordingly, any unabsorbed cellulose will result in inflammation of the tissue surrounding the cellulose.

Various oxidizing agents exist that when combined with cellulose material create oxidized cellulose. These agents typically comprise aqueous hypochlorite salts. However, it has been found that aqueous hypochlorite salts tend to degrade cellulose fibers. When cellulose fibers are placed in aqueous hypochlorite salts for more than one hour, the fibers usually crumble apart, a problem that is exacerbated upon drying. Furthermore, one hour of reaction time does not create the degree of carboxylation necessary to impart adequate hemostatic properties to the fiber. Such degradation is believed to be due to the alkalinity of the hypochlorite solutions rather than to the oxidation process by the hypochlorite ion.

What is needed is a hemostatic device with sufficient material cohesion that creates a more solid and retentive coagulum plug and that can be placed to fill or cover wounds. Based on the foregoing, it is the general object of the present invention to provide a hemostatic device that overcomes the problems and disadvantages of prior art hemostatic devices.

### SUMMARY OF THE PRESENT INVENTION

In one aspect, the present invention is directed to a hemostatic agent made from oxidized cellulose fiber. The oxidized cellulose has a carboxylation content increased by the action of nitrogen dioxide on virgin cellulose fiber. A composition may be incorporated into the oxidized cellulose fiber to cause a pharmacological effect on a wound to which the hemostatic agent is applied. When applied, the oxidized cellulose fiber causes blood emanating from the wound to clot while delivering the composition to the wound. The oxidized cellulose fiber can either be resorbed into the wound or removed from the wound after healing.

In another aspect, the present invention is directed to a hemostatic device. This device comprises a pellet of unwoven oxidized cellulose fiber implantable into a wound. The oxidized cellulose fiber has a carboxylation content that is increased by the action of nitrogen dioxide on virgin cellulose fiber. The oxidized cellulose fiber may also have a composition incorporated therein that is releasable into the wound to provide pharmacological effects to the wound. Upon implanting the pellet into the wound, the oxidized cellulose fiber causes blood emanating from the wound to clot.

In yet another aspect, the present invention is directed to a method of arresting a flow of blood emanating from a wound. In the method, unwoven oxidized cellulose fiber is packed into or placed against a bleed site. The unwoven oxidized cellulose powder may have a composition incorporated therein for release to the wound and to provide a pharmacological effect on the wound. The unwoven oxidized cellulose fiber is produced by, inter alia, exposing the unwoven cellulose fiber to nitrogen dioxide. This exposure provides for an increased carboxylation content that causes the unwoven oxidized cellulose fiber to be more effective at causing hemostasis.

In yet another aspect, the present invention is directed to a method of fabricating oxidized cellulose. In this method, nitrogen dioxide gas is generated in a first vessel and piped or otherwise transferred to a second vessel containing cellulose fibers. The second vessel is purged with an excess amount of nitrogen dioxide gas and sealed. Allowing the second vessel to remain sealed for a predetermined period of time increases the carboxylation content of the cellulose fibers. The oxidized cellulose fibers are subsequently degassed to remove any residual nitrogen dioxide.

In yet another aspect, the present invention is directed to a bandage that can be applied to a bleeding wound. The bandage includes a pad of unwoven oxidized cellulose fibers mounted on a substrate. A composition may be incorporated into the oxidized cellulose fibers. Upon applying the bandage to a bleeding wound, the oxidized cellulose fibers cause blood emanating from the wound to clot.

The devices of the present invention find utility in numerous applications, for example, in tooth extractions where the resulting wound is in the form of a socket. When used to treat tissue wounded as a result of tooth extractions, the devices can be applied to bleeding sockets to promote hemostasis or to sockets in anticipation of the development of dry socket conditions. Furthermore, the devices of the present invention can be used with success as retro-fill material in apicoectomies (root end surgeries). Additionally, patients undergoing blood anticoagulating therapy utilizing warfarin are not required to discontinue their warfarin medications because the clotting mechanism initiated by the oxidized cellulose proceeds via an alternate pathway.

One advantage of the present invention is that medications or other compositions can be incorporated (e.g., imbedded) into the oxidized cellulose. These medications or other compositions can then be dispersed throughout the entire blood clot instead of only on the outer exposed surface thereof. As a result of medications being imbedded into the oxidized cellulose and hemostatic properties of the oxidized cellulose devices, the composition is dispersed three-dimensionally in the wound gap once a soft plug has been formed. This is a superior method of positioning medications within wounds, instead of merely treating wounds in a topical fashion through the barrier of the clot, as the medication is contained within the wound itself and is intimately involved with the healing process. By dispersing the medication directly into the soft plug and the wound, the medication can prevent infection, stimulate cells that are crucial in the healing process, promote healing by reducing the time that is usually required, promote adhesion of the medicine to hard tissues such as bone, and promote adhesion to soft tissue such as mucosa.

Another advantage of the present invention is that in embodiments in which medications or other compositions are incorporated into the oxidized cellulose, the rate of release of such compositions can be controlled. The release can be made to be gradual (or uniform, depending on the type of treatment) and dictated by the healing sequence. For example, as the healing process progresses, the oxidized cellulose device (e.g., a pellet) decreases in size, and the concentration of composition at the inner portions of the device can be made to be less than the concentration of composition at the outer portions of the device, thereby causing less composition to be released over time. This is due to the oxidized cellulose being a three-dimensional network of unwoven fibers. As a body into which the oxidized cellulose (with composition incorporated therein) initiates the healing process, the release of the composition into the soft plug that is in immediate contact with damaged tissues can be made to keep pace with the organization of the clot. Compositions that can be incorporated into the oxidized cellulose include, but are not limited to, antibiotics, bone stimulating drugs, corticosteroids, bone morphogenic proteins, osteoblast-stimulating drugs, odontoblast-stimulating drugs, and any and all other compositions that promote and/or accelerate healing or prevent infection, individually and in combination. Other compositions that may not accelerate healing but may aid in patient comfort and compliance may also be incorporated. Such compositions include, but are not limited to, anesthetics, analgesics, and other drugs that stimulate nerves such as menthol, eucalyptus, and the like.

### BRIEF DESCRIPTION OF THE DRAWING

The FIGURE is a perspective view of a hemostatic healing bandage having an oxidized cellulose pad mounted on a substrate.

### DETAILED DESCRIPTION OF THE INVENTION

The present invention resides in agents for providing blood clotting functions to wounds and devices incorporating such agents. The agents and devices comprise three-dimensional networks of unwoven fiber material. The fiber material is a cellulose-based non-synthetic material that is oxidized and that can be absorbed into biological tissue. The cellulose fiber is preferably a long-chain polymeric polysaccharide derived from cotton and is hereinafter referred to as virgin cellulose. The term “virgin cellulose” as used herein means cellulose that is not hydrolyzed and that is not fragmented at molecular linkages that produce aldehydes or ketones upon being oxidized. Thus, the oxidized cellulose of the present invention includes substantially no aldehydes or ketones. The present invention is not limited to cellulose derived from cotton, however, as the cellulose may be derived from other sources.

Oxidized cellulose, also known as cellulosic acid, absorbable cellulose, or polyanhydroglucuronic acid, is a chemically oxidized form of common cellulose fiber. Oxidized cellulose is cellulose in which the carboxylation content is increased relative to cellulose fiber that has not been oxidized. The increased carboxylation is a result of a variation in the degree of oxidation. The degree of carboxylation can be estimated by the time it takes to dissolve oxidized cellulose in dilute alkaline solutions, such as 0.1-0.5 molar sodium hydroxide. In contrast, cellulose fibers that are not in oxidized form are not soluble in dilute alkaline solutions. Preferably, the carboxylation content is increased up to about 5% relative to the cellulose fiber that has not been oxidized.

One method of manufacturing the oxidized cellulose of the present invention is to expose the cellulose fiber to nitrogen dioxide gas. One method of creating nitrogen dioxide gas is the action of manganese dioxide or manganese disulfide on concentrated nitric acid. The action of manganese dioxide or manganese disulfide on nitric acid is catalytic, and any amount of nitrogen dioxide can be created by the metered addition of nitric acid to the manganese dioxide or manganese disulfide. During this reaction there is also a significant formation of dinitrogen tetroxide which does not interfere in the oxidation process.

Another method of creating nitrogen dioxide is via the action of formaldehyde on concentrated nitric acid. This reaction is not catalytic, and the formaldehyde is consumed in the reaction. The nitrogen dioxide gas is suitable for oxidizing the cotton fibers to the desired degree of oxidation. The degree of oxidation is time dependent, i.e., dependent upon the time the nitrogen dioxide gas is in contact with the fibers.

A preferred method of manufacturing oxidized cellulose via the reaction of cellulose with nitrogen dioxide is to introduce unaltered virgin cellulose in single strand fiber form into a first reaction vessel, while in a second enclosed vessel concentrated nitric acid is metered into manganese dioxide powder. The nitrogen dioxide gas that is evolved in the second vessel is then vented to the first vessel containing the unaltered cellulose. This first vessel is then purged entirely with excess amounts of nitrogen dioxide and left sealed for 2-6 weeks. This may alternatively be done in a pressurized environment of nitrogen dioxide at a pressure of more than one atmosphere. Furthermore, increasing the temperature in the pressurized chamber will increase the pressure thus accelerating the oxidation process. The resulting oxidized cellulose is sufficiently carboxylated to establish rapid local hemostasis when placed onto a bleeding wound. Also, the action of the nitrogen dioxide on the virgin cellulose fiber minimizes the formation of fragments that produce aldehydes and ketone moieties. The oxidized cellulose can also be degassed without washing to provide suitable material for formation into pellets. The resulting pellets can further be gamma-radiated before patient use to provide a sterile material. The gamma radiation does not affect the oxidized fibers and therefore does not negatively affect the hemostatic properties.

After oxidation of the cellulose, one or more compositions capable of producing a pharmacological effect on a wound can be incorporated into the oxidized cellulose. One method of incorporating a composition into the oxidized cellulose comprises imbedding the composition into the cellulose. When the composition is in the form of particlized material, the particles can be introduced into the fibrous matrix of the cellulose. Adhesion of the particles on the cellulose can be the result of one or more mechanisms, e.g., coulombic forces, physical means, and inherent tackiness of either or both the cellulose and the composition itself. Powders can be physically forced into the fibrous network and trapped (suspended) in the interstices defined by the strands of the matrix. Furthermore, compositions in liquid form can be absorbed into the fibers for subsequent delivery to wounds.

Another method of incorporating a composition into oxidized cellulose involves depositing a composition onto the cellulose by applying solublized or slurried composition to the cellulose. Once the solvent of the solution or the carrier of the slurry is removed, the composition remains on the cellulose. The solvent or carrier can be removed using any suitable method including, but not limited to, evaporation, flash drying, vacuum drying, and drainage. Solvents such as alcohols, chlorinated hydrocarbons, liquid hydrocarbons, and the like can be utilized to soak or deliver the compositions into the fibers. Drying can be controlled so that the composition is absorbed only at the immediate surface of the fiber. More specifically, a “surface coat” of medication can be applied onto the fiber.

It should be understood, however, that the compositions do not necessarily need to be applied to the cellulose and absorbed into the fibers or adsorbed onto the surfaces of the fibers for devices fabricated from the composition-laden oxidized cellulose to work. In particular, the oxidized fibers can be soaked in a solution or slurry of composition to facilitate the application of the composition.

The opposite is also possible. The fibers could be soaked in any given medication, followed by a quick washing of the fibers in any solvent that would dissolve the medication back out of the fiber. By controlling the contact time of the solvent, only the medication on the outermost portions of the fibers will be removed leaving the medication on the innermost portions intact. The present invention is not limited in this regard, however, and other methods of incorporating medications into the oxidized cellulose devices are within the scope of this disclosure.

The composition incorporated into the oxidized cellulose may be any one or a combination of various drugs. The various drugs can be imbedded into the oxidized cellulose. Such drugs include, but are not limited to, antibiotics, bone stimulating drugs (AC-100 or Dentonin), corticosteroids, pain suppressing medications, anti-inflammatory drugs, anti-viral drugs, anti-fungal drugs, homeopathic remedies, bone morphogenic proteins, osteoblast-stimulating drugs, odontoblast-stimulating drugs, and any and all other drugs that promote and accelerate healing, reduce pain, prevent infection, whether individually or in combination. Furthermore, proven beneficial materials such as calcium hydroxide powder, mineral trioxide aggregate (MTA), or bioactive glasses can be incorporated into the pellets by means of mechanical trituration, resulting in a three-dimensional network of oxidized cellulose fibers and the particles of aforementioned materials. These, upon hemostasis initiated by the oxidized cellulose fibers, become imbedded and are part of the blood clot and produce beneficial results during the organization of the blood clot. A similar action is to be expected of the above-referenced drugs being entrapped in a three-dimensional network of fibers, their release keeping pace with the organization of the clot. Another method would be the binding of drugs to nanoparticles which are then incorporated in the fiber network allowing them to bond to the fibers. Release of these drugs from the oxidized cellulose may be sustained to keep pace with the healing process of the blood clot.

The type of drug administered to the device can be made site-specific. For instance if bone healing is the objective of the drug delivery, such as can be promoted by means of AC-100, then placement of a pellet or gauze which incorporates this peptide will allow for a slow release of the drug while at the same time stimulating osteoblasts that are responsible for the formation of bone matrix. If an antibiotic is incorporated in a soft tissue wound, the beneficial action of the antibiotic will reduce or eliminate inflammatory reactions that interfere with healing or prevent healing. The beneficial action of the drugs that are incorporated in the fiber mesh is based on the structure of the mesh, namely, as a result of the mesh being composed of a three-dimensional network of unwoven natural fibers.

Oxidized cellulose can be shaped or configured into many useful forms such as a compressible pellet, gauze sheet, porous sponge, thin unwoven sheet, unwoven pad, loose fibrous ball, or meshed pad. In any form, the oxidized cellulose is gently packed in the wound or wound gap to help increase retention by exerting an outward pressure, or it can be placed over the wound.

A hemostatic healing bandage is also possible by applying an oxidized cellulose pad onto an impermeable strip. Referring to the FIGURE, such a hemostatic healing bandage is shown at 10 and is hereinafter referred to as “bandage 10.” Bandage 10 comprises a pad 12 mounted to the impermeable strip, which is shown as a flexible substrate 14, that can be applied to a wound (for example, using a pressure-sensitive adhesive 16 to adhere the bandage 10 to the skin of a wearer). The substrate 14 is a plastic or a cloth member that is conducive to being retained on the skin of an injured person or animal on or proximate a bleeding wound. Particularly if the substrate 14 is a non-breathable plastic material, the substrate may include holes 18 to allow for the dissipation of moisture evaporating from the skin surface. The pad 12 is stitched, glued, or otherwise mounted to the substrate 14 to form the bandage 10. A composition may be incorporated into the oxidized cellulose of the pad 12, such a composition being any of those described above.

A practitioner appreciates devices that improve the efficacy and ease of use of any treatment. Oxidized cellulose is observed to be particularly useful for filling wound gaps when it is compressed into a pellet. A pellet made of loose fibers is compressible and therefore can be easily inserted into a socket. This is ideal when attempting to fill a wound gap and it is desirable that the pellet remains intact throughout the initial stages of healing until the eventual adsorption by the body removes the pellet. The meshed pad can cover wounds, establish hemostasis while at the same time it can release a single drug, or a plurality of drugs, either immediately or by means of a slow release mechanism. The use of pellets or gauze can also be realized in orthopedic surgery where hemostasis can be combined with drugs that suppress infections and stimulate hard and soft tissue formation, thus promoting healing.

Surface wounds can be addressed by the application of a drug-laden sheet of oxidized cellulose gauze, a porous oxidized cellulose sponge, or thin unwoven oxidized cellulose sheet. These devices can be pressed into the surface wound resulting in immediate hemostasis and a deeper penetration of medications into the wound. Depending on the application the device can either be left in place or removed. When left in place the device is physiologically resorbed by the body.

There are multiple clinical applications for oxidized cellulose devices imbedded with drugs or medications. In the dental field it is indicated for treating any bleeding soft tissues, tooth extraction sockets, in periodontal surgery, in apicoectomy cases, in implant dentistry, to fill the space created after cyst removal, to deliver drugs after bone surgery, to deliver drugs that promote healing of pulp after pulp exposures, in pulpotomies and all other clinical cases in dentistry and medicine in which hemostasis is required with the added benefit of delivering drugs for the purpose of controlling infections and the acceleration of healing. In the medical field, pellets or meshed pads can be used for traumatic accidents causing an immediate cessation of bleeding or in any surgical cases in which bleeding needs to be controlled. In veterinary medicine a compressible pellet, gauze sheet, porous sponge, thin unwoven sheet, or meshed pad may be used to control bleeding in animals. The infiltration of medication throughout the wound is especially advantageous in a less than ideal barnyard environment. It is also suitable for minor wounds or scratches that bleed, or such wounds that warrant a simple bandage. Hemophiliac patients and patients with bleeding problems due to blood thinning medication can effectively be treated with the invention.

The oxidized cellulose pellets can be delivered by means of various techniques which will depend on size and location of the area that requires hemostasis. Direct placement in dental extraction sockets, bone openings for implant placement, apicoectomies, and removal of fibromas or cysts are examples where placement is accomplished through direct vision of the defect. Indirect placement can be accomplished by means of endoscopy or laparoscopy using attachments that are known and commonly used by a person skilled in the art.

The quality of oxidation of the cellulose material can be determined by including a cotton string of known strength during the manufacture of the oxidized cellulose. The strength of the string is determined before it is included in the manufacturing process. One method of determining the strength of the string involves attaching a piece of the string to span between two points (e.g., a span of about 3 inches to about 4 inches), incrementally adding weight to the center point of the span, and noting the amount of weight required to cause the string to break. A mean value is obtained over a predetermined number of trials. In another method, the strength of the string can be determined via a pull test using a commercially available strength testing apparatus.

After determining the strength of the string, a length of this string that is sufficient for a predetermined number of pull tests is incorporated into the material being treated to become oxidized cellulose. After completion of the treatment process and further upon completion of analysis of the desired properties of the oxidized cellulose, the strength test of the string is repeated. A mean value is obtained over a predetermined number of trials and compared to the strength of the string before being incorporated into the material being treated to become oxidized cellulose. Subsequent production batches can be made to include the same (untreated) string material, which should be tested after completion of the treatment process. Upon testing the oxidized cellulose, the weight to break the string incorporated into the oxidized cellulose is preferably within about 10% of the mean value of the untreated string.

Prior to sterilization and use, gases that cause oxidation are removed from the oxidized cellulose (whether in the form of pellets, gauze, or other configurations). Strips made of potassium iodide can be used to determine whether such oxidizing gases have been removed. These strips oxidize easily because in the presence of oxidizing agents potassium iodide converts to elemental iodine, which causes a color change from a non-oxidized clear strip to a strip with a brown-red color. If no change in color takes place the final product is free from residual oxidizers.

### EXAMPLE 1

### Comparison of Speeds of Hemostasis

When compared to the ORC of the prior art (Surgicel), the oxidized cellulose of the present invention exhibited a tendency to produce clotting effects significantly faster. For example, in blood clot testing performed using a prothrombin test (PTT), the ORC did not establish hemostasis after 10 minutes, whereas the oxidized cellulose of the present invention established hemostasis after 4.3 minutes. Furthermore, it was noted that the ORC gelled to form a false clot against which the actual clotting took place, while the oxidized cellulose of the present invention absorbed blood to immediately produce a clot.

The foregoing results were confirmed in tests during non-survival surgery performed on pigs. A large incision (1.5 inches long and 0.5-0.75 inches deep) was made in the spleen of a pig. Rapid hemostasis was achieved with the oxidized cellulose of the present invention, whereas the ORC appeared to be ineffective (after 10 minutes, the Surgicel did not clot the blood).

### EXAMPLE 2

### Comparison of Resorbability

The resorbability of the oxidized cellulose of the present invention was determined using an implantation test performed on baboons. Apicoectomies (root end surgeries) were performed on the baboons. Small pellets of the oxidized cellulose were implanted to provide hemostasis at the root ends. No traces of fibers of the oxidized cellulose were present after 120 days of healing, and the bone surrounding the retrofilled material (the oxidized cellulose pellets) displayed normal anatomical histological features.

### EXAMPLE 3

### Comparison of Acidity Values

The acidity values of both the oxidized cellulose of the present invention and the ORC (Surgicel) were measured and compared. In determining the acidity values, both the oxidized cellulose of the present invention and the ORC reached pH values of about 3.5 to about 3.9. The difference in the values, however, is noted with regard to time. The ORC reached pH 3.5 in a few minutes, whereas the oxidized cellulose of the present invention reached pH 3.5 after about an hour.

### EXAMPLE 4

### Carboxylation Testing

Carboxylation testing was carried out according to standard U.S.P. (United States Pharmacopeia) methods. Three (3) experimental materials and one (1) control material were tested to determine the percentage of carboxyl groups on the oxidized cellulose. The loss of carboxyl groups resulting from the drying of the oxidized cellulose was also measured.

| | Treatment time | Carboxylation | Loss of carboxylation |
|---|---|---|---|
| Sample No. | (days) | content (%) | on drying (%) |
| 1 | 7 | 2.9 | 2.2 |
| 2 | 14 | 3.0 | 2.1 |
| 3 | 21 | 3.2 | 1.7 |

The control material comprised Surgicel. Carboxylation of the control was found to be 22.0%. There was no loss of carboxylation content upon drying of the control.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A hemostatic agent, comprising:
oxidized cellulose fiber having a carboxylation content increased relative to cellulose fiber that has not been oxidized, said increased carboxylation content being increased by the action of nitrogen dioxide on virgin cellulose fiber; and
wherein application of said oxidized cellulose fiber to a wound causes blood emanating from said wound to clot; and
wherein said oxidized cellulose fiber is resorbable into said wound.

2. The hemostatic agent of claim 1, wherein said nitrogen dioxide is generated by the catalytic reaction of at least one of manganese dioxide and manganese disulfide with nitric acid.

3. The hemostatic agent of claim 1, wherein said nitrogen dioxide is generated by the reaction of formaldehyde with nitric acid.

4. The hemostatic agent of claim 1, wherein said oxidized cellulose fiber comprises unwoven strands.

5. The hemostatic agent of claim 4, wherein said unwoven strands define a three-dimensional network.

6. The hemostatic agent of claim 1, wherein said virgin cellulose fiber is derived from cotton.

7. The hemostatic agent of claim 1, further comprising a composition incorporated into said oxidized cellulose fiber, said composition having a pharmaceutical effect on said wound.

8. The hemostatic agent of claim 7, wherein said composition is selected from the group consisting of antibiotics, bone stimulating drugs, corticosteroids, pain suppressing medications, anti-inflammatory drug, anti-viral drugs, anti-fungal drugs, homeopathic remedies, bone morphogenic proteins, osteoblast stimulating drugs, odontoblast stimulating drugs, compositions that accelerate healing, pain reducers, infection preventives, calcium hydroxide powder, mineral trioxide aggregate, bioactive glasses, and combinations of the foregoing.

9. The hemostatic agent of claim 7, wherein said composition incorporated into said oxidized cellulose fiber is imbedded into said oxidized cellulose fiber.

10. The hemostatic agent of claim 7, wherein said composition incorporated into said oxidized cellulose fiber is entrapped in a three-dimensional network of said oxidized cellulose fiber.

11. The hemostatic agent of claim 7, wherein said composition incorporated into said oxidized cellulose fiber is bound to nanoparticles which are incorporated into a three-dimensional network of said oxidized cellulose fiber.

12. The hemostatic agent of claim 1, wherein said carboxylation content is increased up to about 5% relative to said cellulose fiber that has not been oxidized.

13. The hemostatic agent of claim 1, wherein said action of said nitrogen dioxide on said virgin cellulose fiber minimizes a formation of fragments that produce aldehydes and ketone moieties.

14. The hemostatic agent of claim 1, wherein said nitrogen dioxide gas is produced by the action of formaldehyde on nitric acid.

15. A hemostatic device, comprising:
a pellet of unwoven oxidized cellulose fiber implantable into a wound, said oxidized cellulose fiber having a carboxylation content increased by the action of nitrogen dioxide on virgin cellulose fiber; and
wherein upon implanting said pellet into said wound, said oxidized cellulose fiber causes blood emanating from said wound to clot.

16. The hemostatic device of claim 15, wherein said nitrogen dioxide is generated by the catalytic reaction of at least one of manganese dioxide and manganese disulfide with nitric acid.

17. The hemostatic device of claim 15, wherein said nitrogen dioxide is generated by the reaction of formaldehyde with nitric acid.

18. The hemostatic device of claim 15, further comprising a composition incorporated into said oxidized cellulose fiber for release into said wound, said composition having a pharmacological effect on said wound.

19. The hemostatic device of claim 18, wherein said composition is selected from the group consisting of antibiotics, bone stimulating drugs, corticosteroids, pain suppressing medications, anti-inflammatory drugs, anti-viral drugs, anti-fungal drugs, homeopathic remedies, bone morphogenic proteins, osteoblast stimulating drugs, odontoblast stimulating drugs, compositions that accelerate healing, pain reducers, infection preventives, calcium hydroxide powder, mineral trioxide aggregate, bioactive glasses, and combinations of the foregoing.

20. The hemostatic device of claim 18, wherein said release of said composition is a sustained release.

21. The hemostatic device of claim 18, wherein said composition is attached to said oxidized cellulose fibers.

22. The hemostatic device of claim 18, wherein said composition is entrapped in a three-dimensional network of said oxidized cellulose fibers.

23. The hemostatic device of claim 18, wherein said composition is bound to nanoparticles which are then incorporated into a three-dimensional network of said oxidized cellulose fibers.

24. The hemostatic device of claim 15, wherein said oxidized cellulose fiber is resorbable into the tissue of said wound.

25. The hemostatic device of claim 15, wherein said oxidized cellulose fiber is removable from the tissue of said wound subsequent to the clotting of said blood.

26. The hemostatic device of claim 15, wherein said action of said nitrogen dioxide on said virgin cellulose fiber minimizes a formation of fragments that produce aldehydes and ketone moieties.

27. A method of arresting a flow of blood emanating from a wound, said method comprising the steps of:
providing unwoven oxidized cellulose fiber, said oxidized cellulose fiber having a carboxylation content increased relative to cellulose fiber that has not been oxidized, said increased carboxylation content being increased by the action of nitrogen dioxide on virgin cellulose fiber;
applying said unwoven oxidized cellulose fiber to said wound, thereby causing hemostasis to result.

28. The method of claim 27, further comprising incorporating a composition into said unwoven oxidized cellulose fiber for release into said wound.

29. The method of claim 28, wherein said composition is selected from the group consisting of antibiotics, bone stimulating drugs, corticosteroids, pain suppressing medications, anti-inflammatory drugs, anti-viral drugs, anti-fungal drugs, homeopathic remedies, bone morphogenic proteins, osteoblast stimulating drugs, odontoblast stimulating drugs, compositions that accelerate healing, pain reducers, infection preventives, calcium hydroxide powder, mineral trioxide aggregate, bioactive glasses, and combinations of the foregoing.

30. The method of claim 28, further comprising allowing said unwoven oxidized cellulose fiber to be resorbed into the tissue of said wound.

31. The method of claim 28, further comprising removing said unwoven oxidized cellulose fiber from said wound.

32. A method of fabricating oxidized cellulose, said method comprising the steps of:
generating nitrogen dioxide gas in a first vessel;
piping said nitrogen dioxide gas to a second vessel containing cellulose fibers;
purging said second vessel with an excess amount of said nitrogen dioxide gas;
sealing said second vessel and allowing said second vessel to remain sealed for a predetermined period of time to increase a carboxylation content of said cellulose fibers; and
degassing said oxidized cellulose fibers.

33. The method of claim 32, further forming said oxidized cellulose fibers into pellets.

34. The method of claim 33, further comprising gamma-radiating said pellets.

35. The method of claim 32, further comprising incorporating a composition into said oxidized cellulose fibers, said composition having a pharmacological effect on a wound to which said oxidized cellulose fibers are applied.

36. A bandage applicable to a bleeding wound, said bandage comprising:
a substrate;
a pad of unwoven oxidized cellulose fibers mounted on said substrate, said oxidized cellulose fiber having a carboxylation content increased relative to cellulose fiber that has not been oxidized, said increased carboxylation content being increased by the action of nitrogen dioxide on virgin cellulose fiber;
wherein applying said pad to blood emanating from said wound causes said blood to clot and wherein said composition provides a pharmacological effect to said wound.

37. The bandage of claim 36, wherein said substrate includes holes to allow for the dissipation of moisture evaporating from a skin surface to which said bandage is applied.

38. The bandage of claim 36, further comprising a composition incorporated into said oxidized cellulose fibers of said pad for delivery to said wound.

39. The bandage of claim 38, wherein said composition is selected from the group consisting of antibiotics, bone stimulating drugs, corticosteroids, pain suppressing medications, anti-inflammatory drugs, anti-viral drugs, anti-fungal drugs, homeopathic remedies, bone morphogenic proteins, osteoblast stimulating drugs, odontoblast stimulating drugs, compositions that accelerate healing, pain reducers, infection preventives, calcium hydroxide powder, mineral trioxide aggregate, bioactive glasses, and combinations of the foregoing.
